(19) United States
(12) Patent Application Publication
(10) Pub. No.: US 2007/0260187 A1
(43) Pub. Date: Nov. 8, 2007

(54) Adjustable sealing catheter anchor

(76) Inventor: Charles Ralph Hawkins, Bartlett, TN (US)

Correspondence Address: CHARLES F.RYE, PATENT ATTORNEY, FIFTH FLOOR, 8 SOUTH THIRD STREET, MEMPHIS, TN, 38103, US

(21) Appl. No.: 11/417,416

(22) Filed: May 4, 2006

(51) Int. Cl. A61M 5/32

(52) U.S. Cl. 604/174

(57) ABSTRACT

An adjustable insertion length entry sealing anchor as used with cannulas. All non-orifice catheters require an entry which is an injury to the recipient. That injury site has the same potential for infection, drainage and other complications as a non beneficial injury. A catheter entry site must accommodate relatively unrestricted movement of the catheter to allow its accurate placement and reduce the probability of further trauma to surrounding tissue during manipulation. The cooperation of the surrounding tissue's natural elasticity and a tapered, stepped or contoured sealing means inserted into the entry site provide a seal that will tend to lessen the opportunity for contamination of or drainage from the entry site. The elasticity of the surrounding tissue also promotes a healing time that is comparable to an insertion without a sealing means and is especially better than a site that is compromised by an infectious or noxious agent. The control of drainage also increases recipient comfort and decreases the presence of growth media for pathogenic agents. This is most beneficial to catheter applications which require the catheter to remain in the recipient for an extended time. It is also often further desirable to customize the catheter's exterior length for the comfort of the recipient, ease of use and to reduce snagging. The present invention provides for entry site sealing by a tapered, stepped or contoured slideably engaged generally winged anchor that further accommodates customization of the catheters length or other manipulations without compromising the proper positioning of the distal tip.

### CROSS REFERENCE TO RELATED APPLICATIONS

NOT APPLICABLE

### STATEMENT REGARDING FEDERALLY SPONSORED RESEARCH OR DEVELOPMENT

NOT APPLICABLE

### REFERENCE TO SEQUENCE LISTING, etc

NOT APPLICABLE

### BACKGROUND OF THE INVENTION

Devices to removeably secure cannulas to the recipient are known in the art. These range from cannula gripping and securing devices for rigid needles to flexible tubes inserted after or while the entry is created. The securing devices range from simple wing on anchor designs to elaborately adjustable platforms. Securing these devices to the recipient is usually accomplished by stitching, pre-applied adhesive on the device, simply taping or a combination. The current invention device and method address adjusting the insertion length, sealing the insertion site, and may accommodate customizing the external length.

U.S. Pat. No. 3,589,361 issued to Loper in 1971 teaches a flexible wing assembly that provides a needle controlling means when the wings are pinched together and a securing means when the wings are affixed to tissue near the insertion site. U.S. Pat. No. 4,006,744 issued to Steer in 1977 teaches a device to couple a cannula tube to a fluid source tube. U.S. Pat. No. 4,192,305 issued to Seberg in 1980 is an example of a relatively short reach catheter with securing wings that accommodates a removable insertion needle. U.S. Pat. No. 4,366,817 issued to Thomas in 1983 teaches a catheter tube anti kinking hub stem and an angle setting riser on the wing section.

U.S. Pat. No. 4,411,654 issued to Boraini in 1983 teaches a splitable introduction catheter with an anti rupture safety sleeve that becomes a catheter insertion site anchor sleeve after the intentional removal of the introduction catheter. U.S. Pat. No. 5,578,013 issued to Bierman in 1996 teaches an elaborate self adhesive securing pad that engages the fluid interconnect and supports a tube clip.

U.S. Pat. No. 4,728,322 issued to Walker in 1988 identifies that a removable catheter inserter is generally larger than the catheter which invites bleeding and increased the chance of infection after the inserter is removed. Walker addresses these complications by use of a cannula that will increase its outer diameter due to the environment it is used in. Walker also utilizes a hub that accommodates the cannula expanded diameter and provides wing anchors.

Although prior patents have advanced the art in both function and ease of use none provide the simplicity of form and function addressed by the present invention device and method to the issues of adjusting the cannula insertion length, securing at and sealing the insertion site and accommodating customizing the external cannula length.

### BRIEF SUMMARY OF THE INVENTION

The present invention is related to establishing a cannula insertion length, generally a catheter, in relation to its insertion site, securing it to the recipient proximal to its insertion site, sealing the insertion site and accommodating customizing its external length if desired. Many catheter applications require that the inserted distal end be positioned in a predetermined location in order to achieve the desired therapeutic result and have specific function tips. The practitioner utilizing the catheter uses various methods to guide the catheter into the desired position. Transluminal applications are the most common however, duct, cavity, in tissue and other applications would benefit from the invention.

A very high percentage of catheters are placed within an existing lumen structure of a recipient. Normally the catheter is introduced into a lumen structure from a remote insertion site and passively or actively guided to the desired location. Once the distal tip is at the desired location of a lumen structure the most common position fixing of the catheter is by externally securing it to the recipient thereby controlling its insertion length. Even catheters and other cannulas that are less position critical would benefit from securing them at their insertion site and especially sealing the insertion site. The invention allows the sealing/securing means to be adjusted on the catheter's length thereby setting its insertion length. The sealing/securing means further provides a sealing section which is partially inserted into the entry site which in conjunction with the elasticity of surrounding tissue tends to seal the insertion. The securing means, which may be unitary with the sealing means, provides the means to removeably secure the device to the recipient. The exterior length of the catheter may be customized by cutting and attaching a connection end to the catheter, generally a hub receiver, which may be the sealing/securing means itself. Especially during long term use the viability of the original catheter to fluid supply connection may become an issue. The customized exterior length may also leave some excess exterior length for cutting the old connection off and attaching a new fluid supply connection thereby minimizing the need to disturb the catheter. The stabilization means may be generic or special formed to generally conform to the area surrounding the intended insertion site.

### BRIEF DESCRIPTION OF THE SEVERAL VIEWS OF THE DRAWING

FIG. 1 shows a side view of the entry sealing means.

FIG. 2 shows an end view of the stabilization means.

FIG. 3 shows a top view of a catheter passed through the entry sealing means engaged into the stabilization means.

FIG. 4 shows a side view of a catheter passed through the entry sealing means engaged into the stabilization means.

FIG. 5 shows a side view of a sealing means engaged into a stabilization means which is formed to hold the sealing means at an angle to the attachment surface.

FIG. 6 shows a side view of a catheter inserted into a vein with the sealing portion of the sealing means partially inserted through the skin, tissue and into the vein with the sealing means engaged into the stabilization means which is attached to the skin.

### DETAILED DESCRIPTION OF THE INVENTION

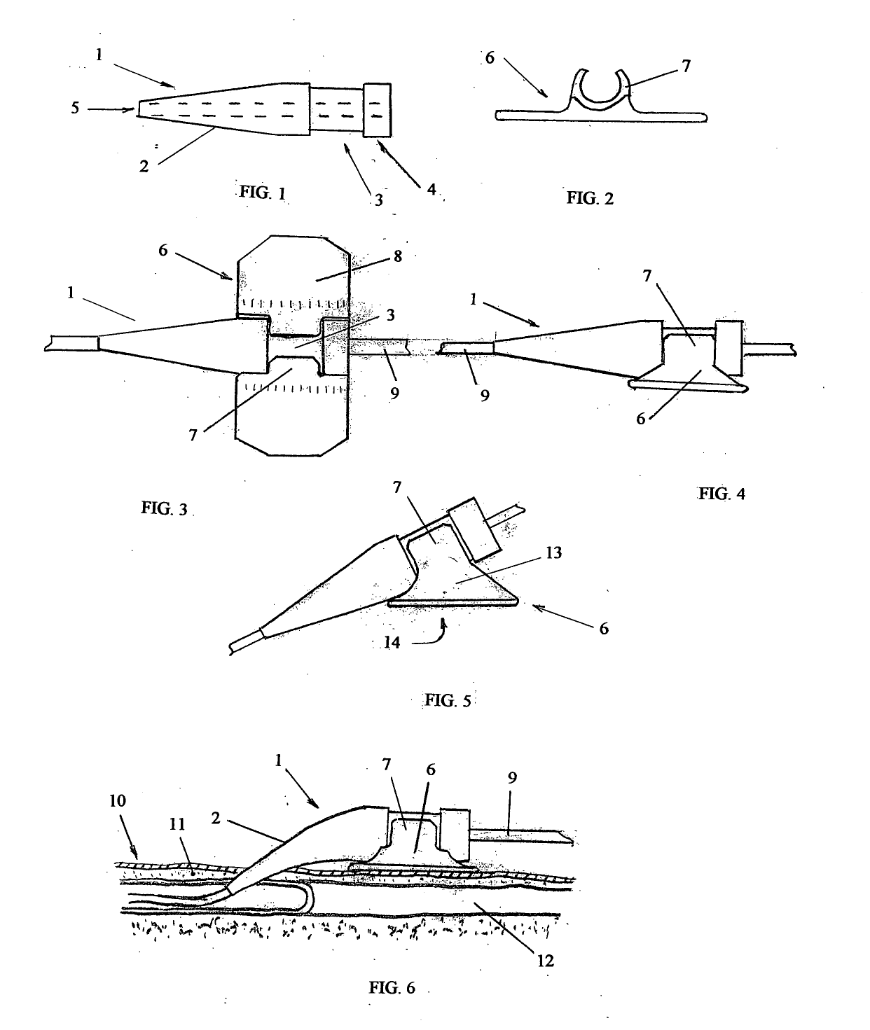

FIG. 1 shows a side view of entry sealing means 1 with pliable tapered sealing section 2, reduced profile middle section 3, opposite end 4 and lumen 5.

FIG. 2 shows an end view of stabilization means 6 with open top resilient barrel grip 7.

FIG. 3 shows a top view of stabilization means 6 with wings 8, and sealing means 1 engaged in barrel grip 7 by way of reduced middle section 3 and with catheter 9 inserted there through.

FIG. 4 shows a side view of stabilization means 6, and sealing means 1 engaged in barrel grip 7 and with catheter 9 inserted there through.

FIG. 5 shows a side view of another embodiment of stabilization means 6 with a raised section 13 which angles barrel grip 7 relative to attaching surface 14.

FIG. 6 shows a side view of stabilization means 6 attached to skin 10, and sealing means 1 engaged in barrel grip 7 and with catheter 9 inserted there through with tapered tip 2 and catheter 9 inserted through skin 10, tissue 11 and into vein 12.

The device has been singularly illustrated by the drawings of one embodiment with an alternate stabilization means. Those skilled in the art may conceive of many equivalent embodiments. The following claims encapsulate the scope or spirit of the device and method invention.

I claim:

1. A cannula sealing and securing set comprising:
(a) a cannula having an insertion end, a main length and a connection end;
said main length having a substantially uniform outer mould line;
(b) an entry sealing means having a sealing section, middle section, an opposing end and lumen therethrough;
said lumen is adaptive to slideably receive said cannula main length;
said sealing section having a major cross section end proximal to said middle section a length and a minor cross section end distal to said middle section; and
(c) a securing means having a body and stabilization means;
said body is adaptive to engageably receive said middle section of said entry sealing means;
said stabilization means is unitary or affixed to said body and adaptive to and removeably affixable to the general surface proximal to a cannula insertion site;
said securing means structure providing a predetermined advantageous orientation of said cannula to said insertion site.

2. The cannula sealing and securing set of claim 1 further comprising said cannula comprising a flexible catheter.

3. The cannula sealing and securing set of claim 1 further comprising said cannula comprising a PICC line.

4. The cannula sealing and securing set of claim 1 further comprising said sealing section being substantially tapered from said minor cross section distal end to said major cross section proximal end.

5. The cannula sealing and securing set of claim 1 further comprising said sealing section being substantially stepped from said minor cross section distal end to said major cross section proximal end

6. The cannula sealing and securing set of claim 1 further comprising said entry sealing means having a preformed slit defined by normally abutted surfaces in communication with and radiating from said lumen over it length.

7. The cannula sealing and securing set of claim 1 further comprising said entry sealing means having a substantially preformed slit defined by normally abutted surfaces in communication with and radiating from said lumen over its length further defining a unitary peal-able outer mould line seal membrane extending from said minor cross section end distal for a predetermined length of said lumen.

8. The cannula sealing and securing set of claim 1 further comprising said opposing end of said entry sealing means defining a hub receptacle.

9. A PICC line catheter sealing and securing set comprising:
(a) a PICC line catheter;
(b) an entry sealing means having a sealing section, middle section, an opposing end and lumen therethrough;
said lumen is adaptive to slideably receive said PICC line catheter said sealing section having a major cross section end proximal to said middle section a length and a minor cross section end distal to said middle section; and
(c) a securing means having a body and stabilization means;
said body is adaptive to engageably receive said middle section of said entry sealing means;
said stabilization means is unitary or affixed to said body and adaptive to and removeably affixable to the general surface proximal to a PICC line insertion site;
said securing means structure providing a predetermined advantageous orientation of said PICC line to said insertion site.

10. The PICC line catheter sealing and securing set of claim 9 further comprising said entry sealing means having a substantially preformed slit defined by normally abutted surfaces in communication with and radiating from said lumen over its length further defining a unitary peal-able outer mould line seal membrane extending from said minor cross section end distal for a predetermined length of said lumen.

11. The PICC line catheter sealing and securing set of claim 9 further comprising said entry sealing means, said securing means including said stabilization means being a unitary structure.

12. A PICC line catheter sealing and securing set comprising:
(a) a PICC line catheter;
(b) an entry sealing means having a sealing section, middle section, an opposing end and lumen therethrough;
said lumen is adaptive to slideably receive said PICC line catheter said sealing section having a major cross section end proximal to said middle section a length and a minor cross section end distal to said middle section;
said entry sealing means having a substantially preformed slit defined by normally abutted surfaces in communication with and radiating from said lumen over its length further defining a unitary peal-able outer mould line seal membrane extending from said minor cross section end distal to said middle section for a predetermined length of said lumen;
said opposing end of said entry sealing means defining a hub receptacle; and
(c) a securing means having a body and stabilization means;
said body is adaptive to engageably receive said middle section of said sealing means;
said stabilization means is unitary or affixed to said body and adaptive to and removeably affixable to the general surface proximal to a PICC line insertion site;
said securing means structure providing a predetermined advantageous orientation of said PICC line to said insertion site;
said entry sealing means, said securing means including said stabilization means being a unitary structure.

13. The method of inserting a length of a cannula and sealing the insertion site comprising the steps of:
preparing the insertion site of the recipient;
determining the desirable insertion length of a cannula before or after insertion into the recipient;
passing said entry sealing means on said cannula main length by way of said lumen and/or said slit to a predetermined position;
inserting said cannula into said insertion site;
inserting said distal end of said sealing section of said entry sealing means into said recipient's entry site for a predetermined length of said sealing section by moving said cannula and said entry sealing means together or by sliding said entry sealing means further on said cannula while keeping said cannula in a fixed position relative to said recipient;
fixing said entry sealing means on said cannula;
engaging said entry sealing means into said stabilization means if separate; and
affixing said stabilization means proximate to said insertion site.

14. The method of claim 13 further comprising the steps of:
determining the desired external length of said cannula
cutting said cannula proximal external end to the desired length
preparing the newly created connection end of said cannula to receive a connection means; and
engaging said connection means onto said connection end.
